(19) United States
(12) Patent Application Publication
(10) Pub. No.: US 2009/0204161 A1
(43) Pub. Date: Aug. 13, 2009

(54) EXTERNAL DEFIBRILLATOR HAVING AN AUTOMATIC OPERATION OVERRIDE

(75) Inventors: Daniel J. Powers, Issaquah, WA (US); Hans Griesser, Bainbridge Island, WA (US); Teresa Skarr, Windsor, CA (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS, N.V., EINDHOVEN (NL)

(21) Appl. No.: 12/304,282

(22) PCT Filed: Jun. 12, 2007
(86) PCT No.: PCT/IB2007/052218

§ 371 Date: Dec. 11, 2008

(30) Foreign Application Priority Data
Jun. 12, 2007 (IB) PCT/IB2007/052218

(51) Int. Cl. A61N 1/00

(52) U.S. Cl. 607/5

(57) ABSTRACT

A defibrillator and method for delivering defibrillation energy to a patient are described. A patient ECG is analyzed and defibrillation energy is delivered to the patient in response to receiving manual input. The patient ECG is further analyzed while waiting for the manual input and the defibrillation energy is delivered to the patient after a time delay if the manual input is not received.

The invention relates generally to electrotherapy circuits, and more particularly, to external defibrillators having operability in a mode that requires action by a rescuer before delivering a shock to a patient and further having an automatic mode of operation override that automatically delivers the shock to the patient in the event the shock is not manually delivered by the rescuer's action.

Defibrillators deliver a high-voltage impulse to the heart in order to restore normal rhythm and contractile function in patients who are experiencing arrhythmia, such as ventricular fibrillation (“VF”) or ventricular tachycardia (“VT”) that is not accompanied by spontaneous circulation. There are several classes of defibrillators, including manual defibrillators, implantable defibrillators, and semi-automatic and automatic external defibrillators. Semi-automatic and automatic external defibrillators are generally referred to as “AEDs.” AEDs differ from manual defibrillators in that AEDs can automatically analyze the electrocardiogram (“ECG”) rhythm to determine if defibrillation is necessary. In some AED designs, a rescuer is prompted to press a shock button to deliver the defibrillation shock to the patient when ECG analysis concludes that a shock is advised. In others, the AED is designed to analyze the patient's ECG and automatically deliver the defibrillation shock without any action by the rescuer.

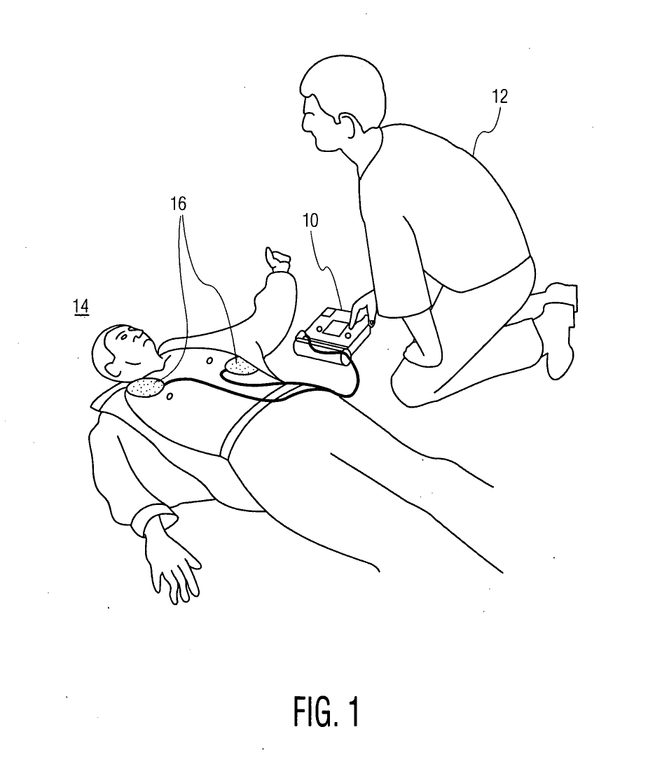

FIG. 1 is an illustration of a defibrillator 10 being applied by a rescuer 12 to resuscitate a patient 14 suffering from cardiac arrest. In sudden cardiac arrest, the patient is stricken with a life threatening interruption to the normal heart rhythm, typically in the form of VF or VT that is not accompanied by spontaneous circulation (i.e., shockable VT). In VF, the normal rhythmic ventricular contractions are replaced by rapid, irregular twitching that results in ineffective and severely reduced pumping by the heart. If normal rhythm is not restored within a time frame commonly understood to be approximately 8 to 10 minutes, the patient 14 will die. Conversely, the sooner circulation can be restored (via a combination of cardio-pulmonary resuscitation “CPR”and defibrillation) after the onset of VF, the greater the chances the patient 14 will survive. The defibrillator 10 may be in the form of an AED capable of being used by a first responder.

A pair of electrodes 16 are applied across the chest of the patient 14 by the rescuer 12 in order to acquire an ECG signal from the patient's heart. The defibrillator 10 then analyzes the ECG signal for signs of arrhythmia. For the case where the defibrillator 10 requires rescuer intervention to deliver the shock, if VF or other shockable rhythm is detected, the defibrillator 10 signals the rescuer 12 that a shock is advised and prompts the rescuer 12 to press a shock button on the defibrillator 10 to deliver a defibrillation pulse to the patient 14.

In treating victims of cardiac arrest with a defibrillator, studies have shown that it is important for treatment to be performed very rapidly because a patient's chances of surviving the cardiac arrest decrease drastically over time following the cardiac arrest. Consequently, the defibrillation shock, if necessary, should be delivered as quickly as possible. Studies have also shown that when CPR precordial compressions are administered to the patient 14, a long pause between cessation of CPR and shock delivery will decrease the patient's chance of survival. Preferably, the delay between CPR and shock delivery is less than 10 seconds.

To facilitate delivery of a defibrillation shock to the patient 14, AEDs have been designed to quickly analyze the patient's ECG, make a determination whether a shock is advisable, and prepare itself for delivery of the shock within a short time period. Preferably the time from when the AED begins analysis of the patient's ECG to the time the AED prompts the rescuer 12 to press the shock button to deliver a shock to the patient 14 is less than 10 seconds. The time in which the AED is ready to deliver a shock is relatively short in part because manual actuation of shock delivery provides a margin of safety by giving notice to the rescuer 12 to stay clear of the patient 14 before delivering a shock. Requiring that the shock button is pressed for shock delivery also forces the rescuer 12 to remove at least one hand from the patient 14 when the shock is delivered, which reduces the likelihood of inadvertently shocking the rescuer 12. Thus, less time following a shock advised prompt is needed to allow a rescuer 12 to stand clear of the patient 14.

A problem with AEDs with this mode of operation, however, is that it requires action from the rescuer 12, namely, pressing the shock button, for a shock to be delivered. Regardless of how quickly the AED is ready to deliver a shock to the patient 14, if the shock button is not pressed, no shock is ever delivered. This situation can arise where an untrained or inexperienced rescuer 12 who is panicked and under great duress because of the emergency situation simply fails to press the shock button when prompted. Even if the rescuer 12 overcomes their anxiety and eventually presses the shock button, the rescuer's hesitation further delays the time that the shock is ultimately delivered to the dying patient 14. Failure to press the shock button can also occur where a patient 14 sensing the onset of cardiac arrest successfully attaches the electrodes 16 to himself or herself, but does not press the shock button because of losing consciousness before the AED completes the ECG analysis or is ready to deliver the shock.

AEDs which deliver the shock without rescuer intervention address the previously described problem by eliminating the need for manual actuation of shock delivery. As previously mentioned, once attached to a patient 14, these AEDs can analyze the patient's ECG and automatically deliver the defibrillating shock. Although solving one problem, AEDs with this mode of operation present problems of their own. For example, to ensure that CPR compressions or patient movement are not interpreted by the AED as a shockable rhythm, the patient's ECG is evaluated over a greater time period before determining that a shock should be delivered. In some cases the time from when ECG analysis begins to when the AED delivers a shock can be between 20 and 30 seconds, and possibly as much as 40 seconds. These times are considerably greater than the 10 second time frame that is preferable. Additionally, to prevent a rescuer 12 from being inadvertently electrocuted, the AED must warn the rescuer 12 to stand clear of the patient 14 and also give the rescuer 12 sufficient time to move away before the shock is automatically delivered. This process further delays when the shock is delivered to the dying patient 14 and further reduces the chances of patient survival.

In accordance with the principles of the present invention a defibrillator is provided having a plurality of electrodes, a high-voltage circuit, a user interface and a controller. The high-voltage circuit is coupled to the electrodes and is operable to develop defibrillation energy to be delivered through the electrodes. The user interface is operable to receive user input. A controller is coupled to the electrodes, the high voltage circuit, and the user interface. The controller is operable to analyze ECG signals for a shockable rhythm and control the high-voltage circuit to deliver defibrillation energy through the electrodes in response to user input. The controller is further operable to further analyze the ECG signals during a time delay and, in response to confirming a shockable rhythm, control the high-voltage circuit to deliver the defibrillation energy after the time delay and/or some other criterion without user input.

Another aspect of the invention provides a method for delivering defibrillation energy to a patient. The method includes analyzing a patient ECG and delivering defibrillation energy to the patient in response to receiving manual input. The method further includes further analyzing the patient ECG while waiting for the manual input and delivering defibrillation energy to the patient after a time delay or other criteria if the manual input is not received.

In the drawings:

FIG. 1 is an illustration of a defibrillator being applied to a patient suffering from cardiac arrest.

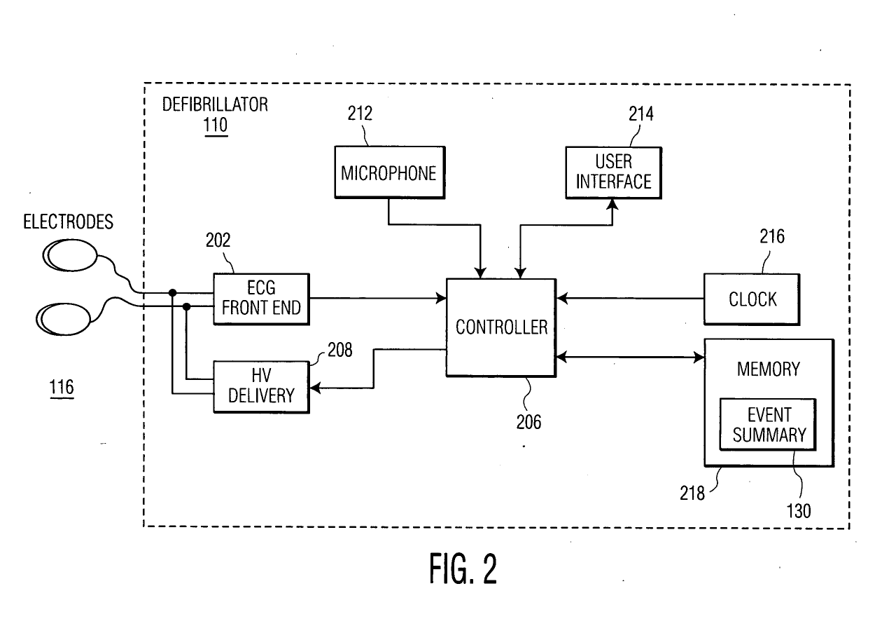

FIG. 2 is a block diagram of a defibrillator constructed in accordance with the principles of the present invention.

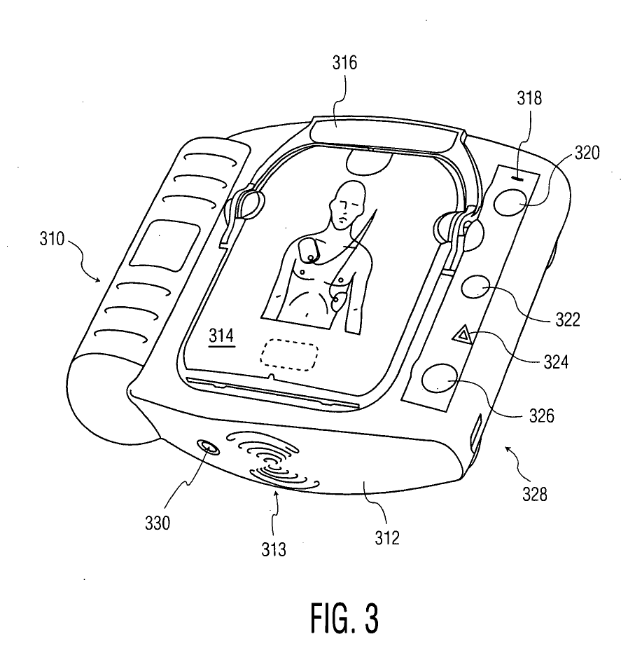

FIG. 3 illustrates an AED with an audible user interface.

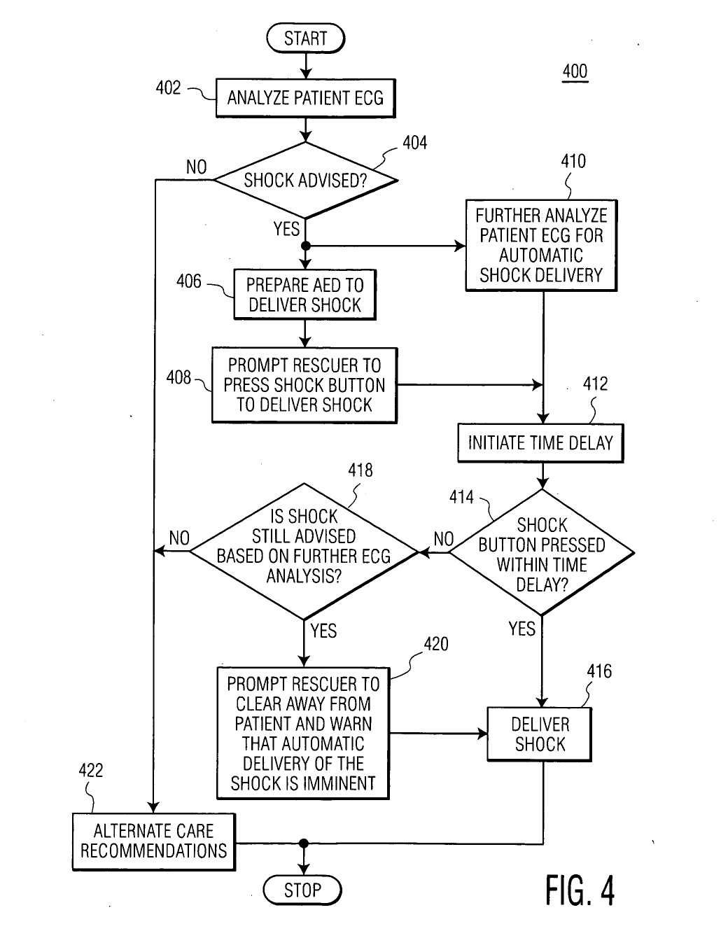

FIG. 4 is a flow chart illustrating the operation steps of the defibrillation system in accordance with an embodiment of the present invention;

Certain details are set forth below to provide a sufficient understanding of the invention. However, it will be clear to one skilled in the art that the invention may be practiced without these particular details. Moreover, the particular embodiments of the present invention described herein are provided by way of example and should not be used to limit the scope of the invention to these particular embodiments. In other instances, well-known circuits, control signals, timing protocols, and software operations have not been shown in detail in order to avoid unnecessarily obscuring the invention.

FIG. 2 illustrates a defibrillator 110 in which an embodiment of the present invention can be implemented. As will be described in more detail below, the defibrillator 110 includes a mode of operation where manual delivery of a shock is initially available followed by automatic shock delivery if manual delivery does not occur. Additional ECG analysis is performed by the defibrillator 110 during the time manual delivery is available to confirm that automatic delivery is appropriate.

An ECG front end circuit 202 is connected to a pair of electrodes 116 that are connected across the chest of the patient 14. The ECG front end circuit 202 operates to amplify, buffer, filter and digitize an electrical ECG signal generated by the patient's heart to produce a stream of digitized ECG samples. The digitized ECG samples are provided to a controller 206 that performs an analysis to detect VF, shockable VT or other shockable rhythm. If a shockable rhythm is detected, the controller 206 sends a signal to high-voltage (“HV”) delivery circuit 208 to charge in preparation for delivering a shock. A shock button on a user interface 214 is activated by the controller 206 to begin flashing to prompt the rescuer 12 that the defibrillator 110 is ready to deliver a shock. When the rescuer 12 presses the shock button on the user interface 214 a defibrillation shock is delivered from the HV delivery circuit 208 to the patient 14 through the electrodes 116. As will be described in greater detail below, if a time delay passes without the shock button being pressed a shock will be automatically delivered to the patient 14.

The controller 206 is coupled to further receive input from a microphone 212 to produce a voice recording. The analog audio signal from the microphone 212 is preferably digitized to produce a stream of digitized audio samples which may be stored as part of an event summary 130 in a memory 218. The user interface 214 may consist of a display, an audio speaker, and control buttons such as an on-off button and a shock button for providing user control as well as visual and audible prompts. A clock 216 provides real-time clock data to the controller 206 for time-stamping information contained in the event summary 130 and can also be used by the controller 206 as a timer to time a time delay. The memory 218, implemented either as on-board RAM, a removable memory card, or a combination of different memory technologies, operates to store the event summary 130 digitally as it is compiled over the treatment of the patient 14. The event summary 130 may include the streams of digitized ECG, audio samples, and other event data as previously described.

Referring now to FIG. 3, an external AED 310 is shown in a top perspective view. The AED 310 is housed in a rugged polymeric case 312 which protects the electronic circuitry inside the case and also protects the rescuer from the hazards of the electrical hardware. The AED 310 includes electronic circuitry similar to that described with reference to FIG. 2 to provide defibrillator operation with an automatic override. Attached to the case 312 by electrical leads are a pair of electrode pads. In the example of FIG. 3 the electrode pads are in a cartridge 314 located in a recess on the top side of the AED 310. The electrode pads are accessed for use by pulling up on a handle 316 which allows removal of a plastic cover over the electrode pads. The user interface is on the right side of the AED 310. A small ready light 318 informs the user of the readiness of the AED 310. In this example the ready light blinks after the AED 310 has been properly set up and is ready for use. The ready light is on constantly when the AED 310 is in use, and the ready light is off or flashes in an alerting color when the AED 310 needs attention.

Below the ready light is an on/off button 320. The on/off button is pressed to turn on the AED 310 for use. To turn off the AED 310 a user holds the on/off button down for one second or more. An information button 322 flashes when information is available for the user. The user depresses the information button to access the available information. A caution light 324 blinks when the AED 310 is acquiring heartbeat information from a patient and lights continuously when a shock is advised, alerting a rescuer and others that no one should be touching the patient during these times. Interaction with the patient while the heart signal is being acquired can introduce unwanted artifacts into the detected ECG signal. A shock button 326 is depressed to deliver a shock after the AED 310 informs the rescuer that a shock is advised. An infrared port 328 on the side of the AED 310 is used to transfer data between the AED and a computer. This data port is typically used after a patient has been rescued and a physician desires to have the patient event data downloaded to his or her computer for detailed analysis. A speaker 313 provides audible instructions to a rescuer to guide the rescuer through the use of the AED 310 to treat a patient. A beeper 330 is provided which “chirps” when the AED 310 needs attention such as electrode pad replacement or a new battery.

FIG. 4 illustrates a process 400 for a manual shock delivery with automatic override mode of operation according to the present invention. The process will be described with reference to the defibrillator 110. However, the process 400 can be performed by other defibrillators as well, including the AED 310 previously described to provide a mode of operation in accordance with the present invention.

At step 402, the controller 206 acquires and analyzes the patient's ECG to determine whether delivery of a shock is advised. If a shock is not advised at step 404, the defibrillator 110 may provide alternate care instructions to the rescuer 12 at step 422, such as administration of CPR, and/or may continue to monitor the patient's ECG waveform. However, if a shockable rhythm is detected, the controller 206 prepares the defibrillator 110 at step 406 to deliver a shock, which can include, for example, the controller 206 directing the HV delivery circuit 208 to charge in preparation for delivering a shock. When the defibrillator 110 is ready to deliver the shock, the rescuer 12 is prompted at step 408, such as by audible or visual prompts, to press the shock button on the user interface 214 to actuate delivery of the defibrillation shock to the patient 14. Concurrently with preparing the defibrillator 110 AED to deliver the shock and prompting the rescuer 12 to manually actuate shock delivery, the controller 206 performs further ECG analysis in the event the shock is to be delivered automatically. The analysis performed by the controller 206 can be conventional ECG analysis for automatic shock delivery as known in the art. For example, the additional analysis can confirm that the patient's ECG is not corrupted by moving or handling the patient and that the ECG amplitude, periodicity and frequency content is indicative of cardiac arrest. Although the additional ECG analysis is described and illustrated as being performed concurrently with steps 406, 408, the additional ECG analysis can be performed sequentially with steps 406, 408 as well.

At step 412 the controller 206 utilizes the clock 216 for measuring a time delay within which the shock button should be pressed by the rescuer 12 to deliver the shock to the patient 14. At step 414, if the shock button is pressed before the time delay elapses, a shock is delivered to the patient 14 at step 416. However, if the time delay elapses and the shock button has not yet been pressed to manually deliver the shock to the patient 14, the controller 206 determines whether a shock is still advised at step 418 based on the further ECG analysis, which preferably continues during the time delay period. If a shock is no longer advised at step 418, alternate care instructions can be provided at step 422 to the rescuer 12. However, if a shock is still advised, the controller 206 issues an audible prompt at step 420 to stand clear of the patient 14 and that a shock will be delivered to the patient 14. The shock is then automatically delivered at step 416 to the patient 14. Although not expressly shown in FIG. 4, a time delay between the audible prompt issued at step 420 and automatic delivery of the shock at step 416 should be provided to allow time for the rescuer 12 to react to the audible prompts and safely move away from the patient 14.

It will be appreciated by those ordinarily skilled in the art that the time delay at step 412 should be selected to provide enough time for a rescuer 12 to react to the visual and/or audible prompts to press the shock button, but short enough to minimize significantly decreasing the likelihood of successful resuscitation. Additionally, the time delay should be sufficiently long to allow for the additional ECG analysis to be completed. In an alternative implementation, the time delay at step 412 can be adjusted by the controller 206. For example, where the ECG analysis indicates that the patient's condition is deteriorating rapidly, the controller 206 can reduce the time delay at step 412 so that the prompts to clear away from the patient 14 and warn the rescuer 12 of imminent shock delivery are issued sooner, and ultimately, the shock can be delivered to the patient 14 sooner. A measure such as a return of spontaneous circulation (“ROSC”) score, can be used to determine patient condition. A more detailed description of calculating a ROSC score is provided in commonly assigned patent application Ser. No. 60/751,269, entitled DEFIBRILLATOR WITH AUTOMATIC SHOCK FIRST/CPR FIRST ALGORITHM, which is incorporated herein by reference. The time delay can also be extended when the ECG analysis indicates that survivability is not compromised by providing additional time for a rescuer 14 to press the shock button.

In alternative implementations of the present invention, the automatic delivery of the shock following expiration of the time delay can be aborted. The abort option gives the rescuer the ability to prevent automatic delivery of the shock in the event there is a reason the shock button was not pressed by the rescuer. The rescuer can be informed of the abort option at the time audible prompts warning of imminent automatic shock delivery are issued. The controller 206 can be programmed to abort the automatic delivery of the shock in response to any of the buttons being pressed.

A mode of operation according to the present invention can be included as a selectable mode of operation in a defibrillator. For example, a defibrillator can be configured by the factory and shipped to customers for rescuer-actuated shock delivery. If an owner such as an emergency medical response service or a potential rescuer prefers the rescuer-actuated shock delivery with automatic override mode of operation, the defibrillator can be programmed to operate in this mode of operation. Various known programming procedures can be used, such as, pressing a button on the user interface multiple times or in another specialized sequence. In response, the controller enters a setup mode and issues audible instructions to announce that the defibrillator is in the setup mode and provide instructions for selecting a mode of operation. By following the audible instructions, the default factory mode of operation can be changed to a rescuer-actuated shock delivery with automatic override mode of operation.

The present invention includes imp lamentations that can be integrated into different conventional treatment protocols to ensure that a shock is delivered to the patient when shock is advised but has not been manually delivered by the rescuer pressing the shock button. For example, in a “s hock first” protocol the defibrillator is set up to immediately analyze the patient's ECG heart rhythm when connected to a patient and activated to make a heart rhythm classification. If the analysis determines that an arrhythmia treatable with electrical defibrillation is present, typically either VF or pulseless VT, the rescuer is informed and the defibrillator is enabled to deliver a shock. A rescuer-actuated shock delivery with automatic override mode of operation according to the present invention ca n be used to automatically deliver the shock if not manually delivered by the rescuer.

Another protocol is a “CPR first” protocol where the defibrillator initially instructs the rescuer to administer CPR to the patient. After CPR is administered for a prescribed period of time, the defibrillator begins to analyze the ECG data to see if an arrhythmia treatable with electrical defibrillation is present. By integrating the rescuer-actuated shock delivery with automatic override mode of operation, if a shock is advised, the rescuer is first given an opportunity to deliver the shock by pressing the shock button. However, as previously discussed, after a time delay elapses, the defibrillator prepares to automatically deliver the shock to the patient.

Embodiments of the present invention can be integrated with other known or later developed treatment protocols as well. For instance, instead of waiting for the expiry of a time delay before delivering the shock automatically, the criterion for automatic shock delivery can be the completion of further analysis or other criteria.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. A defibrillator, comprising:
a plurality of electrodes;
a high-voltage circuit coupled to the electrodes and operable to develop defibrillation energy to be delivered through the electrodes;
a user interface operable to receive user input; and
a controller coupled to the electrodes, the high voltage circuit, and the user interface, the controller operable to analyze ECG signals for a shockable rhythm and control the high-voltage circuit to deliver defibrillation energy through the electrodes in response to user input, the controller further operable to further analyze the ECG signals during a time delay and, in response to confirming a shockable rhythm, control the high-voltage circuit to deliver the defibrillation energy without user input.

2. The defibrillator of claim 1 wherein the user interface comprises a button.

3. The defibrillator of claim 1, further comprising a clock circuit coupled to the controller, the clock circuit operable to time the time delay, wherein the defibrillation energy is delivered following the time delay.

4. The defibrillator of claim 1 wherein the controller comprises a controller further operable to vary the time delay after which the high-voltage circuit is controlled to deliver the defibrillation energy based on the ECG signals.

5. The defibrillator of claim 4 wherein the controller comprises a controller further operable to calculate a score from the ECG signals and vary the time delay based on the score.

6. The defibrillator of claim 1 wherein the user interface comprises an audio system coupled to the controller and the controller comprises a controller further operable to control the audio system to deliver an audible warning that defibrillation energy will be delivered without user input.

7. The defibrillator of claim 6 wherein the controller comprises a controller further operable to control the audio system to deliver an audible prompt to provide user input to deliver defibrillation energy through the electrodes in response to detecting a shockable rhythm.

8. The defibrillator of claim 1 wherein the user interface comprises a plurality of buttons and the controller comprises a controller further operable to abort delivery of the defibrillation energy after the time delay in response to at least one button being pressed.

9. A method for delivering defibrillation energy to a patient, comprising:
analyzing a patient ECG;
delivering defibrillation energy to the patient in response to receiving manual input;
further analyzing the patient ECG while waiting for the manual input; and
delivering defibrillation energy to the patient if the manual input is not received.

10. The method of claim 9, further comprising:
prior to delivering the defibrillation energy to the patient, further analyzing the patient ECG during a time delay; and
providing a warning that defibrillation energy will be delivered without manual input.

11. The method of claim 9, further comprising:
aborting delivery of the defibrillation energy after a time delay in response to receiving manual abort input.

12. The method of claim 9 wherein delivering defibrillation energy in response to receiving manual input comprises delivering defibrillation energy in response to pressing of a button.

13. The method of claim 9 wherein analyzing the patient ECG comprises analyzing the ECG for a shockable rhythm.

14. The method of claim 13 wherein further analyzing the patient ECG comprises further analyzing the patient ECG to confirm the shockable rhythm.

15. The method of claim 9, further comprising developing the defibrillation energy during analysis of the patient ECG.

16. The method of claim 9 wherein delivering defibrillation energy to the patient if the manual input is not received comprises delivering defibrillation energy to the patient after a variable time delay if the manual input is not received, the variable time delay based on the patient ECG.

17. A computer readable medium having computer executable instructions for execution by a programmable controller of a defibrillator for performing a method for delivering defibrillation energy to a patient, the method comprising:
analyzing a patient ECG;
delivering defibrillation energy to the patient in response to receiving manual input;
further analyzing the patient ECG while waiting for the manual input; and
delivering defibrillation energy to the patient after a time delay if the manual input is not received.

18. The computer readable medium of claim 17 further having computer executable instructions for providing a warning that defibrillation energy will be delivered without manual input prior to delivering the defibrillation energy to the patient after the time delay.

19. The computer readable medium of claim 17 wherein analyzing the patient ECG comprises analyzing the ECG for a shockable rhythm.

20. The computer readable medium of claim 19 wherein further analyzing the patient ECG comprises further analyzing the patient ECG to confirm the shockable rhythm.

21. The computer readable medium of claim 17 wherein delivering defibrillation energy to the patient after a time delay if the manual input is not received comprises delivering defibrillation energy to the patient after a variable time delay if the manual input is not received, the variable time delay based on the patient ECG.
